(19) United States
(12) Patent Application Publication
(10) Pub. No.: US 2009/0169497 A1
(43) Pub. Date: Jul. 2, 2009

(54) Treatment and Prevention of Skin Injury Due to Exposure to Ultraviolet Light

(76) Inventors: Lawrence D. Horwitz, Englewood, CO (US); Mayumi Fujita, Englewood, CO (US); David A. Norris, Englewood, CO (US)

Correspondence Address: GREENLEE WINNER AND SULLIVAN P C, 4875 PEARL EAST CIRCLE, SUITE 200, BOULDER, CO, 80301, US

(21) Appl. No.: 12/335,110

(22) Filed: Dec. 15, 2008

Related U.S. Application Data
(60) Provisional application No. 61/018,274, filed on Dec. 31, 2007.

(51) Int. Cl. A61K 8/36; A61K 31/197; A61Q 17/04

(52) U.S. Cl. 424/59; 514/562

(57) ABSTRACT

Photoprotective agents useful for the treatment or prevention of cell damage, particularly skin injury due to UV-light. Methods of prevention or treatment of cell damage and skin injury due to exposure to UV-light, particularly UVB-light. Methods include administration of an effective amount of one or more photoprotective N-mercaptoalkanoylcysteine derivatives of this invention, particularly bucillamine or a salt or solvate thereof, to a mammal, particularly a human, which has been or will be exposed to UV-light. Administration may be oral, topical or parenteral.

### CROSS-REFERENCE TO RELATED APPLICATIONS

This application claims benefit of U.S. provisional application 61/018,274, filed Dec. 31, 2007, which is incorporated in its entirety by reference herein.

### BACKGROUND OF THE INVENTION

The present invention relates to a method for the treatment or prevention of skin injury due to exposure to ultraviolet light using N-mercaptoalkanoylcysteine compounds, particularly bucillamine. These compounds function as photoprotective agents for the management of pathological conditions elicited by UV exposure.

Solar UV radiation is the most prominent and ubiquitous carcinogen in our environment and the skin is its major target Epidemiological, clinical and biological studies have implicated repeated exposures of human skin to solar ultraviolet (UV) irradiation, especially the UVB (290-320 nm) wavelength, as a cause of both melanoma and non-melanoma skin cancers (1-4) Several animal studies have shown that UV radiation can act both as a tumor initiator and a tumor promoter (26-28).

There is considerable evidence that UVB-induced skin cancer is associated with DNA damage (5). DNA damage induces activation of p53 protein and upregulation of p53 transcription. UVB exposure of skin results in various adverse biological responses either through direct (5) or indirect damage to DNA and non-DNA cellular targets via the formation of free radicals, ROS and inflammation (29-31). UVB also causes the generation of reactive oxygen species (ROS) that damage both DNA and non-DNA targets (6-8). Singlet oxygen can be generated as a result of UV absorption by endogenous photosensitive chromophores such as porphyrins and cytochromes (9-11). Newly formed ROS including hydroxyl radical and superoxide anion can activate genes (such as COX-1 which is linked to the inflammatory response), damage DNA or oxidize cell lipids and proteins (7, 12), and thereby modify cellular function (6, 13). Signals transduced from the cell surface to the nucleus through protein phosphorylation at serine/threonine are altered by ROS and redox reactions (14).

Similarly, UVB induces inflammatory responses including edema, dermal infiltration of leukocytes and production of cytokines and growth factors (15, 16). There is mounting evidence that inflammation plays a pivotal role in tumor initiation and promotion (16, 17).

There is a clear need in the art for methods for protecting the skin and more generally mammalian cells against UV-induced damage and particularly against UVB-induced biological responses leading to such damage. Compounds which inhibit UVB-induced ROS generation and inflammation are expected to prevent photodamage and photocarcinogenesis in the skin. Such compounds can also be applied to prevent photodamage and photocarcinogenesis in any mammalian cells, e.g. those of the eyes, exposed to UV light (particularly, but not limited to UVB light). This invention relates to N-mercaptoalkanoylcysteine compounds, particularly bucillamine, which exhibit a photoprotective effect against UV-mediated skin damage.

Several studies have reported that both natural and synthetic antioxidants may have beneficial effects against UV-mediated damage to the intracellular redox state (14). Studies have also shown the beneficial effects of anti-inflammatory drugs against cancer and UV-mediated damage (13, 18). Long-term use of anti-inflammatory drugs, such as aspirin and selective cyclooxygenase-2 inhibitors, significantly reduces cancer risk (18). For example, sulindac, a nonsteroidal anti-inflammatory drug, attenuated UVB-induced inflammatory responses and reduced UVB-induced events relevant to carcinogenesis (13).

Bucillamine, also designated N-(2-mercapto-2-methylpropionyl)-L-cysteine, is a N-mercaptoalkanoylcysteine compound that can replenish endogenous glutathione due to its two donatable thiol groups (19). In addition to its anti-oxidative effect, bucillamine may have an anti-inflammatory effect.

Bucillamine functions as an antioxidant by transferring thiol groups to the endogenous glutathione or thioredoxin systems and maintaining them in a reduced state (32, 33). Animal studies have shown that bucillamine can attenuate tissue damage during myocardial infarction, cardiac surgery, and oxidative injury in reperfusion during organ transplantation (19, 21, 22). Bucillamine can also inhibit DEP-enhanced allergic sensitization in mice and blood-retinal barrier permeability in streptozotocin-induced diabetic rats by reversing ROS production (34-37).

Two closely related compounds, N-acetylcysteine and N-(2-mercaptopropionyl)-glycine, have been shown to work through an antioxidant mechanism (32, 38, 39). All three compounds contain the basic cysteine molecule, but bucillamine has two donatable thiol groups while the other two have only one, which is believed explain the greater potency of bucillamine as an antioxidant. Previously, we have demonstrated the antioxidant mechanism of action of bucillamine in various experimental settings (19, 21, 22).

Bucillamine may also function as an anti-inflammatory drug through effects of an oxidized metabolite. Bucillamine has been used as an effective oral medication in many Asian countries for the treatment of rheumatoid arthritis (RA), a chronic multisystem inflammatory disease (40). Although the mode of action of bucillamine on inflammation is still unclear, it has been shown to inhibit T cell proliferation and production of pro-inflammatory cytokines (41).

N-mercaptoalkanoylcysteines and related compounds have been reported to be useful for various pharmaceutical applications.

U.S. Pat. No. 5,670,545, relates to a method of treatment of ischemic disease and reperfusion injury by administration of bucillamine, N-(2,2-dimethyl-3-mercaptopropionyl)-L-cysteine or related compounds. More specifically, the patent relates to compounds of formula I:

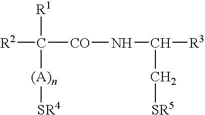

where R¹ and R², independently of one another, can be alkyl groups, particularly lower alkyl groups; R³ can be a carboxyl group or an ester or amide thereof; R⁴ or R⁵, independently of one another, are selected from the group consisting of a hydrogen atom, an alkyl group, an alkanoyl group, a phenyl-alkyl group or a phenylcarbonyl group, and the phenyl ring in the phenyl-alkyl and phenylcarbonyl groups can be substituted by at least one selected from halogen atoms, and alkyl, hydroxy, alkoxy, alkylenedioxy, nitro, amino and alkylamino groups; n is an integer that is 0 or 1; and A is a lower (C1-C6) alkylene group, such as a —CH₂— group. In general these compounds are reported to be useful to prevent damage to living tissue from the formation or presence of reactive oxygen species.

Related U.S. Pat. No. 5,756,547 reports the use of bucillamine, salts thereof and related species in a method for preserving organs or other tissue for transplantation. These patents further state that bucillamine is reported to be useful in a variety of pharmaceutical applications: as a dissolving agent for sputum (see Laid-Open Japanese Patent Application No. 53-5112), an anti-rheumatic agent (see Laid-Open Japanese Patent Application No. 55-51020), a treatment for cataracts (see Laid-Open Japanese Patent Application No. 55-92315, a treatment for diabetes (see Laid-Open Japanese Patent Application No. 4-154721), and a treatment for osteoporosis (see Laid-Open Japanese Patent Application No. 4-154722).

U.S. Pat. No. 5,292,926, relates to certain cysteine derivatives of formula:

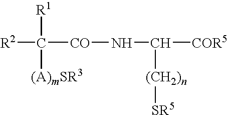

where variables are defined in the patent, which derivatives are reported to be useful as immunomodulators and for treatment of liver disease.

U.S. Pat. No. 4,305,958 relates to N-(2-methyl-2-mercaptoacyl)-cysteine compounds of formula:

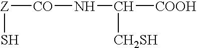

where Z is selected from CH₃CH<, (CH₃)₂C<, —(CH₂)₂— and —CH₂—, which are reported to be useful as agents for the liquefaction of sputum.

U.S. Pat. No. 5,266,595 (November 1993) relates to a method of treatment of cystinuria by administration of an effective amount of bucillamine or a pharmaceutically acceptable salt thereof.

U.S. Pat. No. 6,025,393 (February 2000) relates to methods for treatment of inflammatory intestinal diseases by administration of bucillamine or a salt thereof. Bucillamine is reported to be useful for the treatment inflammatory intestinal diseases and to provide suppressive effect on disorders of the mucous membrane and therapeutic effect on ulcerative colitis. This reference also states that it has been already been found that bucillamine is useful as a therapeutic agent for rheumatic disease, cataract, diabetes, osteoporosis or cystinuria, a dissolving agent for sputum or a suppressive agent for liver disorders and refers to Japanese Patent Publications 11888/1985, 5388/1981, 13922/1987, 13964/1988, and Japanese Unexamined Japanese Patent Publications: 154721/1992, 154722/1922, 342524/1992, 186341/1993).

U.S. Pat. No. 5,688,524 relates to transdermal formulations for administration of prazosin. The formulations described contain prazosin and a skin permeation enhancer composition containing a sulfhydryl-containing compound, a fatty acid ester, and a polar solvent for transdermal administration. The formulations are reported useful to treat hypertension or benign prostatic hypertrophy. The formulation is reported to be employed in the form of a certain skin patch. N-(2-mercaptopropionyl)glycine, thioglycerol, thioacetic acid, thiosalicylic acid, bucillamine, acetylcysteine and mercaptomenthone are reported to be useful non-toxic sulfhydryl-containing compounds.

U.S. Pat. No. 6,197,326 relates to an intra-articular preparation for treatment of arthropathy, such as arthritis and articular rheumatism, comprising microcapsules of certain biodegradable and biocompatible high-molecular weight substances and a drug used as a therapeutic for arthropathy. Among a number of drugs, bucillamine is described as a therapeutic for arthropathy. Examples given of high-molecular substances having biodegradability and biocompatibility are homopolymers and copolymers of lactic acid, glycolic acid, caprolactone, valerolactone, butyrolactone, amino acids, alkyl cyanoacrylates and hydroxybutyrates; albumin; gelatin; starch, casein; and chitosan. U.S. Pat. No. 6,428,804 relates more specifically to injectable formulation of such microcapsule of certain size where the microcapsules are suspended in a dispersing medium comprising at least one compound selected from the group consisting of hyaluronic acid, chondroitin sulfate and salts thereof.

U.S. Pat. No. 6,676,668 relates certain mercaptoacylcysteine derivatives, including bucillamine, as therapeutic agents for osteoarthritis. More specifically, the patent refers to compounds of formula:

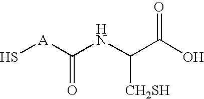

where A is lower alkylene and is exemplified by straight-chain or branched alkylene having one to six carbon atoms such as methylene, ethylene, (dimethyl)methylene or (diethyl)methylene.

U.S. Pat. No. 6,613,807 relates to therapeutic polyanhydride compounds for drug delivery. The polyanhydrides are reported to link low molecular weight drugs containing a carboxylic acid group and an amine, thiol, alcohol or phenol group into polymeric drug delivery systems. Bucillamine is described as an immunosuppressive compound which is a drug that can be delivered through the polyanhydride compounds.

U.S. Pat. No. 6,946,465 relates to immunosuppressive effects of certain pteridine derivatives. The patent also relates to combinations of these immunosuppressive pteridine derivatives with an immunosuppressant or immunomodulator drug, an antineoplastic drug or an antiviral agent, to provide potential synergistic effects. Bucillamine is described, among others, as a suitable immunomodulator drug for use in such combinations.

U.S. Pat. No. 5,821,237 relates to compositions for improving the visual appearance of skin. The compositions described contain certain “primary actives” including at least one cyclic polyanionic polyols, at least one sulfhydryl compound and at least one zwitterionic surfactant. Useful sulfhydryl compounds are said to include N-acetylcysteine, cysteine, glutathione, thioglycolic acid, thioglycolic acid ethyl ester, thiosalicylic acid, cysteamine, dithiothreitol, lipoic acid, dithioerythritol, thioacetic acid, thiolactic acid, mercaptoethanol, dimercaptol, monothioglycerol, N-(2-mercaptoproprionyl)glycine, bucillamine, mercaptomenthone, and combinations thereof.

While there are extensive reports on pharmaceutical applications of N-mercaptoalkanoylcysteine compounds and particularly of bucillamine, there are apparently no reports that such compounds and particularly bucillamine are useful as photoprotective agents and for the treatment or prevention of skin injury due to exposure to ultraviolet light.

### SUMMARY OF THE INVENTION

This invention provides photoprotective agents useful for the treatment or prevention of skin injury due to UV-light. The invention also provides methods of prevention or treatment of skin injury due to exposure to UV-light, particularly UVB-light. The methods of the invention involve the administration of an effective amount of one or more photoprotective N-mercaptoalkanoylcysteine derivatives of this invention to a mammal, particularly a human, which has been or will be exposed to UV-light.

Photoprotective compounds of this invention are those of formula I:

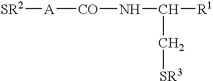

and pharmaceutically acceptable salts and solvates, particularly, hydrates thereof, where R¹ is selected from a carboxylic acid (COOH or COO—) group or an ester (COOR) or amide (CON(R′)₂) thereof, where R is alkyl, phenyl, phenyl-alkyl group (alkylphenyl or phenylalkyl group) and each R′, independently, is H, alkyl, phenyl, phenyl-alkyl group (alkylphenyl or phenylalkyl group); R² or R³, independently of one another, are selected from the group consisting of a hydrogen atom, an alkyl group, an alkanoyl group, a phenyl-alkyl group or a phenylcarbonyl group, wherein the alkyl groups, or the phenyl ring any of these groups is optionally substituted by at least one selected from halogen atoms, alkyl, hydroxy, alkoxy, alkoxyalkyl, alkylene, alkyleneoxy, alkylenedioxy, nitro, amino and alkylamino groups; and A is a lower alkylene group (i.e., a C1-C6 alkylene group) which may be straight chain or branched, such as a CH₃CH<, (CH₃)₂C<, —(CH₃)₂C—CH₂—, —(CH₂)₂— or —CH₂— group.

For all R¹⁻³, R and R′ groups of formula I that contain alkyl, phenyl-alkyl, alkanoyl, alkoxy, alkoxyalkyl, alkylene, alkyleneoxy, alkylenedioxy or alkylamino groups, preferred groups are those that contain from 1 or 2 to about 6 carbon atoms (i.e., lower alkyl).

In specific embodiments, the photoprotective compounds of this invention are those of formula I where R² and R³ are both H. In additional specific embodiments, the photoprotective compounds are those of formula I where A is (R″)₂C<, where each R″, independently, is a C1-C6 alkyl group and more specifically is a methyl, ethyl or propyl group. In additional specific embodiments of the photoprotective compounds of formula I, R² and R³ are both H and A is (R″)₂C<, where each R″, independently, is C1-C6 alkyl group and more specifically is a methyl, ethyl or propyl group. In additional specific embodiments, the photoprotective compounds are those of formula I where A is —(CH₃)₂C—CH₂—, i.e.,

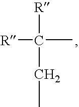

where each R″, independently, is a C1-C6 alkyl group and more specifically is a methyl, ethyl or propyl group. In additional specific embodiments of the photoprotective compounds of formula I, R² and R³ are both H and A is —(CH₃)₂C—CH₂— where each R″, independently, is C1-C6 alkyl group and more specifically is a methyl, ethyl or propyl group. In additional embodiments of the photoprotective compounds of formula I, R¹ is COOH or COO⁻. In specific embodiments, the photoprotective compounds of formula I are derivatives of L-cysteine. In a particular embodiment, the photoprotective compounds are bucillamine and salts and hydrates thereof. In another particular embodiment, the photoprotective compounds are N-(2,2-dimethyl-3-mercaptopropionyl)-L-cysteine and salts and hydrates thereof.

The photoprotective compounds of this invention are optionally employed in the methods herein in combination with a pharmaceutically acceptable carrier. Such carriers include those that are known in the art as carriers suitable for use for administration by oral, topical and parenteral routes.

The photoprotective compounds of this invention may for example be employed in aqueous solutions. The pH in water of certain of the compounds of this invention is acidic, so that it may be necessary to neutralize aqueous solutions with a base, such as NaOH, to preferably achieve physiologic pH.

The photoprotective compounds of this invention can be combined in an effective amount with at least one sunfiltering or sunscreening compound such as para-aminobenzoic acid, salicyclate, cinnamate, benzophenone, anthranilate, dibenzoylmethane, beta-carotene and alpha-hydroxy acids. Such compositions can be employed as improved suncreens for prevention of damage to the skin from UV-light.

The methods, compositions and medicaments of the invention are particularly useful for prevention and treatment of UV-induced acute photodamage, particularly UVB-induced acute photodamage. The methods herein can be employed to treat or protect against UV damage through direct or indirect damage to DNA and non-DNA cellular targets via the formation of free radicals, reactive oxygen species and inflammation.

The invention is also related to medicaments for the treatment or prevention of skin damage due to exposure to UV-light, particularly UVB-light as well as methods for preparation of such medicaments. Such methods involve combination of and effective amount (or combined effective amount) of one or more of the photoprotective agents of this invention with a pharmaceutically effective carrier. The carrier employed is preferably one that is useful for topical or parenteral administration.

The invention additionally relates to the use of one or more photoprotective N-mercaptoalkanoylcysteine derivatives of formula I for treatment or for prevention of skin damage due to exposure to UV-light, particularly UVB-light.

In methods and uses of the invention for prevention of skin damage, the photoprotective compound or a composition containing an effective amount thereof in combination with a pharmaceutically acceptable carrier is administered to the individual in need of such protection prior to exposure to UV-light. The compound or composition is preferably administered 12 hours or less, more preferably 5 hours or less and yet more preferably 2 hours or less prior to exposure to UV-light. In methods and uses of the invention for treatment of skin damage, the photoprotective compound or a composition containing an effective amount thereof in combination with a pharmaceutically acceptable carrier is administered to the individual in need of such treatment after exposure to UV-light. The compound or composition is preferably administered as soon as possible after exposure. Treatment will often be administered when detrimental symptoms of excessive exposure to UV-light are noted.

### DETAILED DESCRIPTION OF THE INVENTION

The term “alkyl group” refers to a monoradical of a branched or unbranched (straight-chain or linear) saturated hydrocarbon. The term as used herein also included cycloalkyl groups. Useful alkyl groups typically contain 1-20 carbon atoms. Preferred alkyl groups have 8 or fewer carbon atoms. The term lower alkyl group indicates alkyl groups having from 1 to 6 carbon atoms and includes for example methyl, ethyl, propyl, pentyl, hexyl, isopropyl, isobutyl, isopentyl, isohexyl, t-butyl and t-pentyl groups. More preferred lower alkyl groups are methyl and ethyl groups.

The term “alkoxy group” (or alkoxide) refers to a —O-alkyl group, where alkyl groups are as defined above. The alkyl group can be linear, branched or cyclic. Again useful alkoxy groups typically contain 1-20 carbon atoms and preferred alkoxy groups have 8 or fewer carbon atoms. The term lower alkoxy indicates alkoxy groups having from 1 to about 6 carbon atoms and includes for example methoxy, ethoxy, propoxy, pentoxy, hexyloxy, isopropoxy, isobutoxy, isopentoxy, isohexyloxy, t-butoxy and t-pentoxy groups. More preferred lower alkoxy groups are methoxy and ethoxy groups.

The terms “alkanoyl” refers to an alkyl-CO— group where alkyl is defined above and refers to straight-chain, branched or cyclic alkyl groups. When this term is modified by the word “lower” it refers to linear, branched or cyclic alkyl groups having from 2 up to about 6 carbon atoms. Exemplary alkanoyl groups include: acetyl, propionyl, butyryl, valeryl, pivaloyl, among others. The more preferred alkanoyl group is acetyl.

The term alkoxyalkyl refers to alkyl groups in which one or more of the carbons are replaced with an —O— atom. These groups can also be referred to as ether groups. The term is intended to encompass groups with one oxygen, such as CₙH₂ₙ₊₁—O—CₘH₂ₘ— as well as groups with two or more oxygens, such as CₙH₂ₙ₊₁—O—CₘH₂ₘ—O—CₚH₂ₚ—, where n, m and p are integers, preferably ranging from 1-6 and more specifically 1, 2, or 3. Exemplary alkoxyalkyl groups include among others methoxymethyl, methoxyethyl, ethoxyethyl, ethoxyethoxyethyl, or butoxyethyl. Alkyl portions of the alkoxyalkyl groups can be straight-chain or branched. In specific embodiments, alkoxyalkyl groups contain straight -chain alkyl portions.

The term “alkylene”, “alkyleneoxy” and “alkylenedioxy” refer to —(CH₂)ₙ— groups (alkylene) and to groups in which one or two (alkyleneoxy and alkylenedioxy, respectively) CH₂ moieties of the alkylene are replaced with an oxygen atom. When these terms are modified by the word “lower” they refer to linear or branched groups having from 1 or 2 (as appropriate for the particular group) up to about 6 carbon atoms. Certain alkylenedioxy groups have a linear or branched alkylene group between two oxygen atoms. Exemplary alkenyl groups include, methylene, ethylene, trimethylene, hexamethylene, propylene, (ethyl)methylene, and (dimethyl)methylene groups. Exemplary alkyleneoxy groups include methyleneoxy (—CH₂—O—), dimethyleneoxy (—CH₂—O—CH₂—), ethyleneoxy (—CH₂—CH₂—O—), diethyleneoxy (—CH₂—CH₂—O—CH₂—CH₂—), propyleneoxy (—CH₂—CH₂—CH₂—O—), etc. Exemplary alkylenedioxy groups include: methylenedioxy (—O—CH₂—O—), ethylenedioxy (—O—CH₂—CH₂—O—), propylenedioxy, and (diethyl)methylenedioxy as well as more complex groups such as (—O—CH₂—CH₂—O—CH₂—CH₂—)

The term phenyl refers to a species of structure:

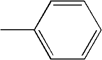

and generally includes the unsubstituted and substituted forms thereof. The term “phenyl-alkyl” refers to a group containing a phenyl and a linear or branched alkyl group. The group may be attached to a core molecule via the alkyl group, such as in a benzyl group (also called an phenylalkyl group, or a phenyl-substituted alkyl group) or via the phenyl group, such as in a (4-methyl)phenyl group (also called an alkylphenyl group, or an alkyl-substituted phenyl group). The phenyl ring in the phenyl-alkyl group can be optionally substituted by a group selected from halogen atoms (F, Cl, or I), lower alkyl, hydroxy, lower alkoxyalkyl, lower alkoxy, lower alkylene, lower alkyleneoxy, lower alkylenedioxy, nitro, amino, and lower alkylamino.

The terms “ester” or “amide” refer to ordinary esters or amides of carboxylic acids. Lower alkyl esters include methyl esters, ethyl esters, isopropyl esters, butyl esters and hexyl esters. Esters include phenyl-alkyl esters, such as benzyl esters. Amides include amides with ammonia; lower alkyl amines either primary or secondary amines, such as methyl amine, diethyl amine, ethyl amine, diethyl amine; and amides with phenyl-alkylamines, such as benzylamine. Preferred esters are methyl, ethyl or benzyl esters. Preferred amides are amides with ammonia or amides with methyl amine, dimethyl amine, ethyl amine or diethyl amine.

The term “amino” group refers generically to a —N(R)₂ group wherein each R independently is hydrogen or an alkyl, phenyl or phenyl-alkyl group as defined above. Amino groups include —NH₂. An “alkyl amino” group refers to an amino group wherein at least one R is alkyl. A “phenyl amino” group refers to an amino group wherein at least one R contains a phenyl group.

The term “salt(s)”, as employed herein, denotes acidic and/or basic salts formed with inorganic and/or organic acids and bases. Pharmaceutically acceptable salts of the compounds of this invention are those acceptable for use in medicines administered to mammals or humans. They include, among many others, salts with alkali metals or alkaline earth metals; ammonium salts; and salts with organic amines such as diethylamine or triethanolamine. In addition, when a compound of formula I contains both a basic moiety, such as, but not limited to an amine and an acidic moiety, such as, but not limited to, a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound of the formula I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Compounds of the present invention, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention.

The invention expressly includes pharmaceutically usable solvates of compounds according to formula I. The compounds of formula I and salts thereof can be solvated, e.g. hydrated. The solvation can occur in the course of the manufacturing process or can take place, e.g. as a consequence of hygroscopic properties of an initially anhydrous compound of formula I (hydration).

The compounds of this invention may contain one or more chiral centers. Accordingly, unless specifically stated, the invention is intended to include the use of racemic mixtures, diasteromers, enantiomers and mixtures enriched in one or more stereoisomer. The scope of the invention as described and claimed encompasses the racemic forms of the compounds as well as the individual enantiomers and non-racemic mixtures thereof. Additionally, inventive compounds may have trans and cis isomers The invention includes all such isomers, as well as mixtures of cis and trans isomers. When no specific mention is made of the configuration (cis, trans or R or S) of a compound (or of an asymmetric carbon), then any one of the isomers or a mixture of more than one isomer is intended. The processes for preparation of compounds of this invention can use racemates, enantiomers, or diastereomers as starting materials. When enantiomeric or diastereomeric products are prepared, they can be separated by conventional methods, for example, by chromatographic or fractional crystallization.

In addition, compounds of formula I may have prodrug forms. As used herein, a prodrug is a compound that, upon in vivo administration, is metabolized or otherwise converted to the biologically, pharmaceutically or therapeutically active form of the compound. Any compound that will be converted in vivo to provide the bioactive agent (i.e., a compound of formula I) is a prodrug within the scope and spirit of the invention. Various forms of prodrugs are well known in the art. For examples of such prodrug derivatives, see: Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191 (1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988) and Nogrady (1985) Medicinal Chemistry A Biochemical Approach, Oxford University Press, New York, pages 388-392). To produce a prodrug, the pharmaceutically active compound is modified such that the active compound will be regenerated by metabolic processes. The prodrug may be designed as known in the art to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

The compounds of this invention of formula I are commercially available or can be prepared by well known techniques or by ready adaptation of such well-known methods from readily available starting materials. U.S. Pat. Nos. 4,305,958 and 5,292,926, for example, provide details of the preparation of certain compounds of this invention.

Treatment and prevention methods of this invention comprise the step of administering an effective amount of one or more compounds of formula I or a salt or solvate thereof, in particular bucillamine or a salt or solvate thereof, to an individual to treat or prevent injury to cells due to exposure to ultraviolet light. More specifically, the compounds of this invention are administered in an effective amount to treat or prevent damage to skin due to UV light. The term “effective amount,” as used herein, refers to the amount of the compound, that, when administered to the individual is effective to at least partially treat or prevent cell damage or more specifically skin damage, or to at least partially ameliorate a symptom of such damage. Prevention includes decreasing damage, minimizing damage and or completely preventing damage. In a specific embodiment, the compounds of this invention are employed in an effective amount to prevent or treat edema, erythema or thickening of the skin and an effective amount is that amount which when administered to the individual which is effective for prevention of the occurrence of, or effective for amelioration of any of edema, erythema or thickening of the skin. Exposure of cells including those of skin and eyes results in various biological responses either through direct or indirect damage to DNA and non-DNA cellular targets via the formation of free radicals, reactive oxygen species and inflammation. Exposure to UV light can damage cells and tissue in various ways as is described herein and as is known in the art.

As is understood in the art, the effective amount of a given compound will depend at least in part upon, the mode of administration, the cite of administration, any carrier or vehicle (e.g., solution, emulsion, etc.) employed, the extent of damage and the specific individual to whom the compound is to be administered (age, weight, condition, sex, etc.). The dosage requirements need to achieve the “effective amount” vary with the particular compositions employed, the route of administration, the severity of the symptoms presented and the particular subject being treated. Based on the results obtained in standard pharmacological test procedures, projected daily dosages of active compound can be determined as is understood in the art. An effective amount of one or more compounds of formula I or salts or solvates thereof can be combined with an effective amount of other active ingredients, such as suncreening or sunfiltering compounds. The effective amount of the compounds of this invention needed in such compositions may be affected by the presence of other active ingredients. One or ordinary skill in the art can readily determine the effective amount of a compound of this invention by a variety of assay systems which are well-known in the art.

Compounds of this invention can be employed in unit dosage form, e.g. as tablets or capsules, or pre-measured liquid doses. In such form, the active compound or more typically a pharmaceutical composition containing the active compound is sub-divided in unit dose containing appropriate quantities of the active compound; the unit dosage forms can be packaged compositions, for example, packaged powders, vials, ampules, pre-filled syringes or sachets containing liquids. The unit dosage form can be, for example, a capsule or tablet itself, or it can be the appropriate number of any such compositions in package form. Compositions of the invention can be administered before, during, or after exposure to sunlight or other sources of UV-light.

The dosage of the compounds of the invention can vary within wide limits and as is understood in the art will have to be adjusted to the individual requirements in each particular case as discussed above. By way of general guidance, the daily oral dosage can vary from about 0.01 mg to 1000 mg, 0.1 mg to 100 mg, or 10 mg to 500 mg per day of a compound of Formula I or of the corresponding amount of a pharmaceutically acceptable salt or solvate thereof. The daily dose may be administered as single dose or in divided doses and, in addition, the upper limit can also be exceeded when this is found to be indicated.

Any suitable form of administration can be employed in the method herein. The compounds of this invention can, for example, be administered in oral dosage forms including tablets, capsules, pills, powders, granules, elixirs, tinctures, suspensions, syrups and emulsions. Oral dosage forms may include sustained release or timed release formulations. The compounds of this invention may also be administered topically, intravenously, intraperitoneally, subcutaneously, or intramuscularly, all using dosage forms well known to those of ordinary skill in the pharmaceutical arts. Compounds of the invention can further be administered topically employing appropriate carriers.

The therapeutically active compounds of the invention can be administered alone, but generally will be administered with a pharmaceutical carrier selected upon the basis of the chosen route of administration and standard pharmaceutical practice.

Administration includes any form of administration that is known in the art and is intended to encompass administration in any appropriate dosage form and further is intended to encompass administration of a compound, alone or in a pharmaceutically acceptable carrier. Administration is intended to encompass administration of a compound, pharmaceutically acceptable salt, solvate or ester thereof alone or in a pharmaceutically acceptable carrier thereof or administration of a prodrug of a compound of this invention which will form an equivalent amount of the active compound or substance within the body.

Pharmaceutical compositions and medicaments of this invention comprise one or more compounds in combination with a pharmaceutically acceptable carrier, excipient, or diluent. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety.

Pharmaceutically acceptable carriers are those carriers that are compatible with the other ingredients in the formulation and are biologically acceptable. Carriers can be solid or liquid. Solid carriers can include one or more substances that can also act as flavoring agents, lubricants, solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Compositions for oral administration can be in either liquid or solid form.

The compounds of this invention can also be administered to the eye, preferably as a topical ophthalmic formulation. The compounds of this invention can also be combined with a preservative and an appropriate vehicle such as mineral oil or liquid lanolin to provide an ophthalmic ointment.

Compounds of this invention can also be administered in intranasal form by topical use of suitable intranasal vehicles. For intranasal or intrabronchial inhalation or insulation, the compounds of this invention may be formulated into an aqueous or partially aqueous solution, which can then be utilized in the form of an aerosol. The compounds of this invention may be administered rectally or vaginally in the form of a conventional suppository.

The compounds of this invention may also be administered transdermally through the use of a transdermal patch containing the active compound and a carrier that is inert to the active compound, is non toxic to the skin, and allows delivery of the agent for systemic absorption into the blood stream via the skin.

The compounds of the invention may be administered employing an occlusive device. A variety of occlusive devices can be used to release an ingredient into the blood stream such as a semipermeable membrane covering a reservoir containing the active ingredient with or without a carrier, or a matrix containing the active ingredient. Other occlusive devices are known in the literature.

Solid or liquid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, nanoemulsions, nanoparticles, liposomes, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water of appropriate purity, aqueous solutions (particularly containing additives as above, e.g. cellulose derivatives, sodium carboxymethyl cellulose solution), alcohols (including monohydric alcohols and polyhydric alcohols e.g. glycols) and their derivatives, and oils. For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant. Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

Suitable pharmaceutical excipients include starch, cellulose, talc, glucose, lactose, sucrose, gelatin, malt, rice, flour, chalk, silica gel, magnesium stearate, sodium stearate, glyerol, monosterate, sodium chloride, dried skim milk, glycerol, propylene glycol, water, ethanol, and the like. The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like.

The carrier can also be in the form of creams and ointments, pastes, gels and hydrogels, particularly for topical application. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable.

In topical administration, in order to permit access or improve access of the active compounds of this invention to cell which need protection or which are damaged, vehicles which improve their penetration through the outer layers of the skin can be employed. Vehicle constituents which improve the penetration of compounds of the invention into the skin include, but are not limited to: ethanol, isopropanol, diethylene glycol ethers such as diethylene glycol monoethyl ether, azone (1-dodecylazacycloheptan-2-one), oleic acid, linoleic acid, propylene glycol, hypertonic concentrations of glycerol, lactic acid, glycolic acid, citric acid, and malic acid.

Other agents which can be incorporated into a topical composition of the invention for administration to the skin include topical anesthetics including but not limited to benzocaine, lidocaine, and benzyl alcohol, aloe vera and aloe barbadensis, retinoids, DNA repair enzymes, antibacterial agents (e.g. quaternary ammonium compounds, bacitracin, neomycin, polymyxin), zinc salts, and methylxanthines. More generally, known active ingredients compatible with the one or more compounds of the invention, which have some utility in treating or attenuating various aspects of skin injury or discomfort can be employed in topical compositions of this invention.

The compounds or compositions of the invention can be applied to the skin in a spray, lotion, roll-on, stick, or gel. Topical compositions of the invention can be applied as a daily-use skin treatment, once to several times per day, especially on sun-exposed parts of the body, or sites of inflammatory skin conditions. Compositions of the invention can be used for treatment of existing lesions due to prior sun exposure, as well as prevention of, or attenuation of the severity of, damage due to present and future exposure to sunlight or other mutagens.

In certain embodiments, the compound of formula I or salts or solvate thereof are combined with sunscreening or sunfiltering compounds. Sunscreen agents include hydrophilic or lipophilic organic UV-A and/or UV-B sunscreen agent such as cinnamate, benzophenone, beta-carotene, and alpha-hydroxy acids.

Topical compositions of the invention comprising sunscreen may also include, in addition, conventional cosmetic adjuvants and additives such as preservatives, organic solvents, browning agents, antioxidants, stabilizers, emollients, silicones, alpha-hydroxy acids, demulcents, anti-foaming agents, moisturizing agents, vitamins, fragrances, ionic or nonionic thickeners, surfactants, fillers, thickeners, sequestrants, polymers, propellants, alkalinizing or acidifying agents, opacifiers, fatty compounds (e.g. oil, wax, alcohols, esters, fatty acids), colorants, or mixtures thereof or any other ingredient that may be used in cosmetics and in particular for the production of sunscreen compositions.

In specific embodiments, the invention provides a method for treatment or prevention of damage to the skin due to exposure to UV-light which comprises the step of administering to an individual susceptible to such damage an effective amount of one or more compounds of formula:

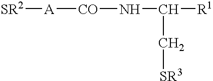

and pharmaceutically acceptable salts and solvates thereof;
where R¹ is selected from a carboxylic acid (COOH or COO—) group, an ester (COOR) or an amide (CON(R′)₂) thereof, where R is alkyl, phenyl, or phenyl-alkyl and each R′, independently, is hydrogen, alkyl, phenyl, or phenyl-alkyl group; R² or R³, independently of one another, are selected from hydrogen, an alkyl group, an alkanoyl group, a phenyl-alkyl group or a phenylcarbonyl group, wherein the alkyl groups, or the phenyl ring any of these groups is optionally substituted with one or more halogen atoms, alkyl, hydroxy, alkoxy, alkylene, alkyleneoxy, alkylenedioxy, nitro, amino or alkylamino groups; and A is a lower alkylene group which may be straight chain or branched.

In more specific embodiments of this method in the compound of formula I, A is a CH₃CH<, (CH₃)₂C<, —(CH₃)₂C—CH₂—, —(CH₂)₂— or —CH₂— group. In other specific embodiments of the method in the compound of formula I both of R² and R³ are hydrogens. In other embodiments of the method in the compound of formula I R¹ is COOH or COO⁻. In specific embodiments, the compound employed in the methods herein is bucillamine or a salt or hydrate thereof.

In additional embodiments, the one or more compounds of formula I of formula I are administered after exposure of the individual to UV-light to treat damage from UV-light.

In any embodiment of the method herein, the compound of formula I can be administered in combination with a pharmaceutically acceptable carrier. In specific embodiments, the carrier can be a sunscreen, such as a commercially available sunscreen containing an amount of a sunscreening or sunfiltering compound sufficient to exhibit useful protection against UVA light, UVB light or both.

In other embodiments of the method of treatment or prevention the one or more compounds of formula I and optional carrier are administered parentally. In yet other embodiments of the methods herein the one or more compounds of formula I and optional carrier are administered topically to skin that has been or may be exposed to UV light. The methods herein can be applied for prevention and treatment of UV-induced acute photo damage. The methods herein can be applied for prevention and treatment of UVB-induced acute photo damage.

This invention additionally relates to the use of one or more of the compounds of formula I above or a salt or solvate thereof in the manufacture of a medicament for treatment or prevention of damage to the mammalian cells, particularly human cells, from UV-light. More specifically, the invention relates to the use of one or more of the compounds of formula I above or a salt or solvate thereof in the manufacture of a medicament for treatment or prevention of damage to the skin of an individual (mammal, including human) from UV-light In specific embodiments, the medicament manufactured is in an oral dosage form as a solid (tablets or pills) a liquid, spray or aerosol, or other appropriate form for oral administration. In specific embodiments, the medicament manufactured is in the form of a liquid, spray or aerosol, cream, gel, or hydrogel or other appropriate form for topical administration to the skin. Additionally, the medicament manufactured is in a form appropriate for ophthalmic administration (liquid, spray, aerosol, cream, gel or hydrogel) to the eyes of an individual in need of protection of treatment In other specific embodiments, the medicament is manufactured in an appropriate vehicle for parenteral administration, particularly by subcutaneous administration, to individuals in need of such protection or treatment. In specific embodiments, the medicament manufactured further comprises a pharmaceutically acceptable carrier or diluent and particularly a carrier or diluent suitable for oral, topical, parenteral, subcutaneous or ophthalmic administration. In specific embodiments, the medicament is manufactured using bucillamine or a salt or solvate thereof in a form suitable for oral, parenteral, topical or ophthalmic administration. In specific embodiments, the medicament manufactured comprises bucillamine or a salt or solvate thereof further comprises a pharmaceutically acceptable carrier or diluent and particularly a carrier or diluent suitable for oral, topical, parenteral, subcutaneous or ophthalmic administration.

In specific embodiments, the invention provides the use of one or more of the compounds of formula I above or a salt or solvate thereof in the manufacture of a medicament for treatment or prevention of damage to mammalian cells, particularly human cells, due to UV-light and particularly for treatment or prevention of damage to the skin or eyes of an individual (mammal, including human) due to UV light. In specific embodiments the medicament manufactured is in an oral dosage form, for example, as tablets or pills. In specific embodiments the medicament manufactured is in a topical dosage form, for example, as a cream, gel or hydrogel. In specific embodiments, the medicament further comprises a pharmaceutically acceptable carrier or diluent and particularly a carrier or diluent suitable for oral, topical or parenteral administration.

The invention also provides a method for treatment or prevention of UV-induced edema, erythema or thickening of the skin which comprises administration of an effective amount of one or more compounds of formula I or a salt or solvate thereof to skin which may be exposed to UV light or to skin which has been exposed to UV light and particularly to skin which exhibits the symptoms of edema, erythema or thickening. In a specific embodiment of this method, the one or more compounds of formula I (or salts or solvates thereof) are administered orally optionally in combination with an appropriate pharmaceutically acceptable carrier for oral administration. In a specific embodiment of this method, the one or more compounds of formula I (or salts or solvates thereof) are administered orally optionally in combination with an appropriate pharmaceutically acceptable carrier for parenteral administration. In a specific embodiment of this method, the one or more compounds of formula I (or salts or solvates thereof) are optionally combined with an effective amount of a sunscreening or sunfiltering compound. An effective amount of a sunscreening or sunfiltering compound is an amount that provides a useful level of protection from UVA light UVB light or both. A large number of sunscreening and sunfiltering compounds are known in the art and one of ordinary skill in the art knows or can readily determine an effective amount of such compounds for use in the methods of this invention. In this method, the compound of formula I or a salt or solvate thereof can be administered parentally or topically optionally in combination with a carrier or a sunscreening or sunfiltering compound.

The invention further provides a medicament comprising an effective amount of one or more compounds of formula I or a salt or solvate thereof for treatment or prevention of UV-induced damage to skin or more specifically for the prevention or treatment of UV-induced edema, erythema or thickening of the skin. In a specific embodiment, the medicament is provided in an oral dosage form for administration to an individual in need of treatment or protection of damage due to UV light. In a specific embodiment, the medicament is provided in topical form and comprises an effective amount of one or more compounds of formula I and an effective amount of at least one sunfiltering or sunscreening compound. The medicament optionally comprises a pharmaceutically acceptable carrier for oral or topical administration. In specific embodiments the compound of formula I in the medicament is bucillamine or a salt or solvate thereof.

The invention also provides a method of manufacture of the medicament for the prevention or treatment of UV-induced edema, erythema or thickening of the skin by combining an effective amount of a compound of formula I or a salt or solvate thereof with a pharmaceutically acceptable carrier for oral, topical or parenteral administration. In a specific embodiment, the medicament manufactured is formulated for topical administration and is combined with an effective amount of a sunscreening or sunfiltering compound and optionally is further combined with a carrier suitable for topical application. The medicament manufactured may also be in a form suitable for ophthalmic administration.

In a specific embodiment, the invention provides an improved sunscreen of sunfilter composition which comprises an effective amount of one or more compounds of formula I. In a more specific embodiment, the sunscreen composition comprises bucillamine or a salt or solvate thereof.

In specific embodiments, the pharmaceutical compositions, dosage forms and/or medicaments of this invention exclude one or more of the following: Prazosin, a fatty acid ester, a polar solvent for transdermal administration, a biodegradable or biocompatible homopolymer, a biodegradable or biocompatible copolymer, homopolymers or copolymers of lactic acid, homopolymers or copolymers of glycolic acid, homopolymers or copolymers of caprolactone, homopolymers or copolymers of valerolactone, homopolymers or copolymers of butyrolactone, homopolymers or copolymers of amino acids, homopolymers or copolymers of alkyl cyanoacrylates, homopolymers or copolymers of hydroxybutyrates; albumin; gelatin; starch, casein; chitosan, hyaluronic acid, chondroitin sulfate or salts thereof, microcapsules, microcapsules suspended in a dispersing medium, microcapsules suspended in a dispersing medium comprising at least one compound selected from the group consisting of hyaluronic acid, chondroitin sulfate or salts thereof, polyanhydride compounds for drug delivery, immunosuppressive pteridine derivatives, cyclic polyanionic polyols, a zwitterionic surfactant, N-acetylcysteine, cysteine, glutathione, thioglycolic acid, thioglycolic acid ethyl ester, thiosalicylic acid, cysteamine, dithiothreitol, lipoic acid, dithioerythritol, thioacetic acid, thiolactic acid, mercaptoethanol, dimercaptol, monothioglycerol, N-(2-mercaptoproprionyl)glycine, or mercaptomenthone.

In other specific embodiments, the pharmaceutical compositions, dosage forms and/or medicaments of this invention can comprise a fatty acid ester, a polar solvent for transdermal administration, a biodegradable or biocompatible homopolymer, a biodegradable or biocompatible copolymer, homopolymers or copolymers of lactic acid, homopolymers or copolymers of glycolic acid, homopolymers or copolymers of caprolactone, homopolymers or copolymers of valerolactone, homopolymers or copolymers of butyrolactone, homopolymers or copolymers of amino acids, homopolymers or copolymers of alkyl cyanoacrylates, homopolymers or copolymers of hydroxybutyrates; albumin; gelatin; starch, casein; chitosan, polyanhydride compounds for drug delivery, immunosuppressive pteridine derivatives, cyclic polyanionic polyols, a zwitterionic surfactant, N-acetylcysteine, cysteine, glutathione, thioglycolic acid, thioglycolic acid ethyl ester, thiosalicylic acid, cysteamine, dithiothreitol, lipoic acid, dithioerythritol, thioacetic acid, thiolactic acid, mercaptoethanol, dimercaptol, monothioglycerol, N-(2-mercaptoproprionyl)glycine, or mercaptomenthone In other embodiments, the pharmaceutical compositions, dosage forms and/or medicaments of this invention can be in the form of microcapsules, microcapsules suspended in a dispersing medium, microcapsules suspended in a dispersing medium comprising at least one compound selected from the group consisting of hyaluronic acid, chondroitin sulfate and salts thereof, or a polyanhydride compound for drug delivery.

When a Markush group or other grouping is used herein, all individual members of the group and all possible combinations and subcombinations of the group are intended to be individually included in the disclosure. Whenever a range is given in the specification, for example, a temperature range, a time range, a composition or concentration range, or a range of variables in a chemical formula, all intermediate ranges and subranges, as well as all individual values included in the ranges given, —are intended to be included in the disclosure.

Molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. Pharmaceutically acceptable salts are preferred for applications herein.

Every formulation or combination of components described or exemplified herein can be used to practice the invention, unless otherwise stated.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. When the broad term “comprising” is used herein, it is intended to encompass the narrower terms “consisting essentially of” and “consisting of.” Thus, the phrase “comprising A and B” encompasses and supports the use of the narrower phrase “consisting essentially of A and B” and the even narrower phrase “consisting of A and B.” The invention illustratively described herein suitably may be practiced in the absence of any element or elements, limitation or limitations which is not specifically disclosed herein.

One of ordinary skill in the art will appreciate that compounds, pharmaceutically acceptable carriers, additives, dosage forms, administration methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference to the extent that there is no inconsistency with the disclosure of this specification. Some references provided herein are incorporated by reference to provide details concerning sources of active compounds of the invention, additional pharmaceutical carriers and additives, additional administration methods and additional uses of the invention.

### THE EXAMPLES

### Example 1

### Assays with Bucillamine—a Representative N-mercaptoalkanoylcysteine

In order to assess any adverse effects of bucillamine and UV exposure separately, a preliminary experiment was carried out in which SKH-1 mice were either treated with bucillamine (20 mg/kg s.c.) or UVB (230 mJ/cm²) irradiation. Bucillamine can, for example, be administered intravenously, subcutaneously, intramuscularly or orally. Subcutaneous administration was used because it is a relatively easy route and the least likely to injure the animal during administration. The selection of bucillamine dose and UVB dose was based on previously published studies (13, 19, 21, 22). High dose of UVB (230 mJ/cm²) was selected to investigate bucillamine effect on acute photodamage. Each group of animals received two doses of bucillamine or UVB, 24 hours apart. Bucillamine was found to have no adverse effects in such experiments. UVB exposure caused edema, erythema and thickening of the exposed skin. Therefore the dose of bucillamine selected for these experiments was found to be safe and tolerable to the SKH-1 hairless mice. In addition the dose of UVB was found to cause mild, discernible skin damage.

### Example 2

### Effects of Bucillamine on Histology in UV-Exposed Skin

UVB exposure induced mild edema, erythema and thickening of the dorsal skin in untreated SKH-1 mice, while bucillamine pre-treatment attenuated the erythema. UV-exposed skin in untreated mice showed scattered necrotic epidermal keratinocytes, papillary dermal edema and dermal infiltration of leukocytes at 6 hours after the last UVB exposure. In bucillamine pre-treated mice there were similar abnormalities at 6 hours after the last UVB exposure, but the epidermal necrosis was less prominent. At 24 hours after the last UVB exposure, UV-exposed skin in untreated mice showed hyperkeratosis and acanthosis (thickening of the epidermis) in the epidermis and papillary dermal edema, infiltration of leukocytes and dilated blood vessels in the dermis. In contrast, bucillamine pre-treatment attenuated the UV-effects on inflammation (dermal edema, leukocyte infiltration and dilatation of blood vessels) at 24 hours after the last UVB exposure.

Effects of Bucillamine on UVB-Mediated p53 Activation.

p53 plays a pivotal role in cellular stress responses. It governs the adaptive and protective responses, following several types of stresses. When normal cells are subjected to stress signals, such as DNA damage or oxidative stress, p53 is activated, resulting in transcription of downstream targets that coordinate cellular growth arrest or apoptosis (23).

UVB exposure resulted in a strong induction of total p53 at 6, 12 and 24 hours after the last UVB exposure, while bucillamine pre-treatment attenuated this effect at 6 hours after the last UVB exposure. p53 level was further decreased at 12 and 24 hours after the last UVB exposure in bucillamine pre-treated mice. Consistent with these findings, immunohistochemical analysis showed increased nuclear p53 staining in the epidermal keratinocytes from UV-exposed skin, whereas bucillamine pre-treatment diminished this effect at 24 hours after the last UVB exposure.

Stabilization and Upregulation of p53 by UVB is Mediated by Phosphorylation but not Inhibition of Degradation.

p53 levels are regulated by protein modifications and proteolytic degradation. In particular, phosphorylation at serine (Ser) 15 and serine (Ser) 20 residues of p53 are known to be essential for stabilization and activation of p53. It is also well documented that p53 is predominantly targeted for destruction by the ubiquitin proteasomal pathway (UPP), and inhibition of UPP results in the upregulation of p53 protein (24). Hence we assessed the effect of UVB and bucillamine on the phosphorylation of p53 and UPP pathway.

UVB exposure at 230 mJ/cm² resulted in strong induction of phosphorylation at both Ser15 and Ser20 at 6, 12 and 24 hours after the last UVB exposure, while bucillamine pre-treatment attenuated this effect at 6 hours after the last UVB exposure and further diminished the phosphorylation at 12 and 24 hours after the last UVB exposure.

UVB exposure did not result in the formation of high molecular weight ubiquitin positive material, a characteristic feature of inhibition of UPP pathway (25). As a positive control of UPP inhibition, we used 8B20 mouse melanoma cells treated with UPP inhibitors MG132 (10 μM) and Lactacystin (10 μM), and as a negative control we used 8B20 cells treated with etoposide (50 μM), a DNA damaging agent. Positive controls showed an increase in the ubiquitin reactive high molecular weight bands between 50 and 250 kD, while a negative control showed no polyubiquitinated species.

Effect of Bucillamine on PUMA, a Downstream Target of p53.

To further investigate the effect of p53 activation by UVB, its downstream target PUMA, a p53-upregulated modulator of apoptosis, was analyzed. The activation of PUMA coincided with the activation of p53 at 6, 12, and 24 hours after the last UVB exposure in UV-exposed skin samples. However, pre-treatment with bucillamine attenuated the activation of PUMA at 12 and 24 hours after the last UVB exposure. Thus, while UVB exposure induced p53 and its downstream target, PUMA, bucillamine treatment inhibited activation of both p53 and PUMA at 12 and 24 hours after the last UVB exposure.

The effect of bucillamine, an anti-oxidant and anti-inflammatory agent and a representative compound of formula I herein, against UVB-induced acute photodamage has been determined, which provides insight into the molecular mechanisms for photoprotection. UV exposure of the dorsal skin of SKH-1 mice induced inflammatory responses and p53 activation as early as 6 hours after the last UVB exposure. Bucillamine pre-treatment attenuated UVB-mediated inflammatory responses and p53 activation at 6 hours after the last UVB exposure and further diminished the effect at 12 and 24 hours after the last UVB exposure.

Bucillamine functions as an antioxidant by transferring thiol groups to the endogenous glutathione or thioredoxin systems and maintaining them in a reduced state (32) (33). Animal studies have shown that bucillamine can attenuate tissue damage during myocardial infarction, cardiac surgery, and oxidative injury in reperfusion during organ transplantation (19, 21, 22). Bucillamine can also inhibit DEP-enhanced allergic sensitization in mice and blood-retinal barrier permeability in streptozotocin-induced diabetic rats by reversing ROS production (34-37). Bucillamine and other compounds of formula I have two donatable thiol groups which are believed to result in greater potency of bucillamine (and other such compounds) as an antioxidant. The antioxidant mechanism of action of bucillamine in various experimental settings has been reported previously (19, 21, 22).

Bucillamine may also function as an anti-inflammatory drug through effects of an oxidized metabolite. Although the mode of action of bucillamine on inflammation is still unclear, it has been shown to inhibit T cell proliferation and production of pro-inflammatory cytokines (41). UV exposure to keratinocytes induces the release of pro-inflammatory cytokines such as IL-1, IL-6, IL-8, IL-10 and TNF-α (42). In particular, IL-1 is a pleiotropic pro-inflammatory cytokine and plays a critical role in cell growth and differentiation, tissue repair, and regulation of immune response by inducing other cytokines (IL-6, IL-18 and TNF-α), growth factors (GM-CSF, VEGF), proteases and inflammatory mediators (COX-2 and iNOS) (43). Inflammatory responses induce ROS at the sites of inflammation, which can lead to further tissue damage associated with inflammation. UVB irradiation induces inflammation clinically and histologically. Pre-treatment with bucillamine before UVB irradiation attenuated the inflammation at 12 and 24 hours after the last UVB exposure.

p53 is regulated by proteolytic degradation and activation of p53. UVB-mediated increase of p53 is not due to the inhibition of UPP proteolytic degradation. Activation of p53 in response to DNA damage involves an increase in p53 protein levels, through stabilization of p53 protein (31, 44, 45). The stabilization of p53 in response to DNA damage has been attributed biochemically to the phosphorylation of p53 serine residues, including Ser-15 and Ser-20 (46, 47). DNA-damaging agents activate phosphorylation of p53 at Ser-15 by a family of protein kinases, including ATM and ATR, and Ser-20 by the Chk2 kinase. These phosphorylations prevent the binding of Mdm2, a negative regulator of p53.

Consistent with these studies, UVB irradiation induces the phosphorylation of p53-Ser-15 and p53-Ser-20 at 6, 12 and 24 hours after the last UVB exposure. However, pre-treatment with bucillamine before UV irradiation resulted in a quick decrease in the activation of p53-Ser-15 and p53-Ser-20 at 12 and 24 hours after the last UVB exposure despite the activation at 6 hours after the last UVB exposure. These results indicate that bucillamine protects against UVB-mediated p53 activation and UVB-mediated p53 phosphorylation. An alternative explanation, is that bucillamine facilitates UPP-dependent or UPP-independent degradation process of p53.

Further, bucillamine has been demonstrated to have an inhibitory effect on the activation of PUMA, a downstream target of p53. It is well known that DNA damage such as UV exposure can cause p53-mediated cell cycle arrest and/or apoptosis. PUMA is a BH3-only protein which is an essential trigger for the induction of apoptosis by binding to the anti-apoptotic protein Bcl-2 (48).

Pre-treatment of SKH-1 mice with bucillamine before UVB irradiation has a protective effect to quickly attenuate the inflammatory responses and p53 activation via phosphorylation of Ser-15 and Ser-20 residues of p53 protein. Bucillamine also has an inhibitory effect on the activation of a downstream target of p53, PUMA, a key pro-apoptotic molecule.

### Example 3

### Materials and Methods for Examples 1 and 2

Chemicals and Reagents. Powdered bucillamine (>99% purity) was obtained from Keystone Biomedical, Inc. (Los Angeles, Calif.). Stock solutions of bucillamine (10 mg/ml) were made in normal saline, pH adjusted to approximately 7.4 with equimolar NaOH, and filter sterilized before injecting into the animals. Anti-actin (mouse monoclonal) was purchased from Sigma (St. Louis, Mo.). Anti-p53 (rabbit polyclonal) was obtained from Novocastra (UK). Phospho-p53 (ser15) and phospho-p53 (ser20) antibodies were from Cell Signaling Technology (Danvers, Mass.). Anti-ubiquitin antibody was a rabbit polyclonal from Sigma (St. Louis, Mo.), while anti-PUMA rabbit polyclonal was purchased from Cell Signaling (Danvers, Mass.). Horseradish peroxidase-conjugated secondary antibodies were purchased from Jackson Immuno Research Laboratories, Inc. (West Grove, Pa.). Complete Mini (protease inhibitor cocktail tablets, cat #11 836 153 001) was obtained from Roche Diagnostics (Mannhein, Germany) and added to lysis buffer for skin lysate preparation. All chemicals and reagents used in this study were of highest purity grade available commercially.

Animals and treatment for Example 2. Female SKH-1 hairless mice (6 weeks old) were purchased from Charles River Laboratories (Wilmington, Mass.). After their arrival, animals were housed at UCDHSC vivarium under specific pathogen-free conditions according to the National Institutes of Health Animal Care Guidelines under an Institutional protocol reviewed and approved by the Institutional Animal Care and Use Committee (IACUC). Animals were allowed to acclimatize for a week prior to the experiment. Animals were fed Purina chow diet and water ad libitum. Throughout the experimental protocol, the mice were maintained at standard conditions; temperature 24±2° C., relative humidity 50±10%, and 12 hour room light/12 hour dark cycle. Mice were divided into four groups. The first group of six mice did not receive any exposure or treatment and served as control (Group 1). The remaining animals were divided into UV exposure alone (Group 2), UV+saline treatment (Group 3) and UV+bucillamine treatment (Group 4). Mice in groups 2-4 were exposed to two doses of 230 mJ/cm² UVB, 24 hours apart. Two hours prior to each UVB exposure, mice in groups 3 and 4 were treated with normal saline (Group 3) or bucillamine at a concentration of 20 mg/kg of body weight (Group 4) subcutaneously. Animals were sacrificed after the last UVB exposure at various time points, and the dorsal skin was surgically removed and used for further analysis.

UVB Source. We employed four FS-40-T-12-UVB sunlamps with UVB spectra 305 Dosimeter (Daavlin, Bryan, Ohio), which emitted about 80% radiation in the range of 280-340 nm with a peak emission at 314 nm as monitored with a SEL 240 photodetector, 103 filter and 1008 diffuser attached to an IL1400A NIST Traceable Radiometer/Photometer from International Light (Newburyport, Mass.). The UVB irradiation doses were also calibrated using an IL1400A radiometer.

Histopathology. Skin samples were fixed in 10% formalin and embedded in paraffin. Vertical sections (4 μm thickness) were cut and mounted on a glass slide, and stained with hematoxylin and eosin followed by microscopic evaluation of the slides. SKH-1 hairless mice were unexposed or irradiated with UVB (230 mJ/cm²) twice, 24 hours apart. Mice irradiated with UVB were untreated or pre-treated with bucillamine or saline. Skin samples were collected at 6 hours and 24 hours after the last UVB exposure, and processed for histopathological analysis.

Immunohistochemical analysis. Formalin-fixed and paraffin-embedded samples were sectioned by microtome, heat immobilized and deparaffinized using xylene, and rehydrated in a graded series of ethanol with a final wash in distilled water. Antigen retrieval was achieved by boiling the sections in citric acid buffer (pH 6.0) in a microwave oven (at 650 W) until the solution boiled. At this time, the power of the microwave was lowered to 100 W for 15 minutes. The samples were allowed to cool at room temperature followed by rinsing with PBS. The tissue sections were then subjected to incubation with 3% H₂O₂ in methanol for quenching the endogenous peroxidase activity. Non-specific binding sites were blocked by incubating with PBS containing 1% BSA and 0.1% Tween 20 for 10 minutes. Sections were incubated with the appropriate primary antibody for 1 hour at 25° C., followed by appropriate biotinylated secondary antibody for 1 hour at room temperature and conjugated horseradish peroxidase (HRP) streptavidin for 45 minutes in a humid chamber. Color development was achieved by incubation with 3,3′-diaminobenzidine for 10 minutes at room temperature. The sections were counterstained with Harris hematoxylin, dehydrated and mounted for microscopic observation. Immunohistochemical analyses were done using Zeiss Axioscop 2 microscope (Carl Zeiss, Inc., Jena, Germany). All samples were coded and evaluated by at least two investigators in a blinded manner. Pictures were taken by Kodak DC 290 camera and processed by Kodak Microscopy Documentation System 290 (Eastman Kodak Company, Rochester, N.Y.).

Preparation of Epidermal Skin Lysate. Total Tissue Lysates were Prepared as described previously (20). Briefly, mouse skin samples were homogenized in ice-cold lysis buffer (50 mM Tris-Hcl, 150 mM NaCl, 1 mM EGTA, 1 mM EDTA, 20 mM NaF, 100 mM Na₃VO₄, 0.5% NP-40, 1% Triton X-100 and 1 mM PMSF (pH 7.4) with a freshly added protease inhibitor cocktail (Roche Diagnostics, Mannhein, Germany). The homogenate was centrifuged at 13,000 rpm for 15 minutes at 4° C., and the supernatant (total cell lysate) was collected, aliquoted and stored at −80° C. Protein analysis was carried out using the Bio-Rad assay kit as per the manufacturer's protocol. BSA was used as a standard for the protein measurements.

SDS-PAGE and immunoblot analysis. Proteins were analyzed by SDS-PAGE (12% gels) followed by transfer to a polyvinylidene difluoride membrane (0.4 μM) in 25 mM Tris, 192 mM glycine, and 20% methanol at 110 V for 1 hour. Membranes were blocked overnight in 10 mM Tris-HCl, pH 8.0, 125 mM NaCl, and 0.2% (v/v) Tween 20 containing 5% (w/v) nonfat dry milk (TBST-M5). Immunoblot analysis of the total protein bands were visualized using enhanced chemiluminescence as described by the manufacturer (Pierce Biotechnology, Inc., Rockford, Ill.). Immunoblots were subsequently stripped of antibodies by incubating for 15-20 minutes at 37° C. with Restore Western Stripping Buffer (Pierce Biotechnology, Inc., Rockford, Ill.) and re-probed with β-actin antibody to confirm protein loading. Densitometric analysis of the membranes was performed using GelDoc 200 (Bio-Rad Laboratories, Hercules, Calif.).

### REFERENCES

1. Ananthaswamy, H. N., and W. E. Pierceall. (1990) Molecular mechanisms of ultraviolet radiation carcinogenesis. Photochem Photobiol. 52:1119-1136.
2. Johnson, T. M., O. M. Dolan, T. A. Hamilton, M. C. Lu, N. A. Swanson, and L. Lowe. (1998) Clinical and histologic trends of melanoma. J Am Acad Dermatol. 38:681-686.
3. Miller, D. L., and M. A. Weinstock. (1994) Nonmelanoma skin cancer in the United States: incidence. J Am Acad Dermatol. 30:774-778.
4. Urbach, F. (1991) Incidence of nonmelanoma skin cancer. Dermatol Clin. 9:751-755.
5. Mitchell, D. L., R. Greinert, F. R. de Gruijl, K. L. Guikers, E. W. Breitbart, M. Byrom, M. M. Gallmeier, M. G. Lowery, and B. Volkmer. (1999) Effects of chronic low-dose ultraviolet B radiation on DNA damage and repair in mouse skin. Cancer Res. 59:2875-2884.
6. Pathak, M. A., and K. Stratton. (1968) Free radicals in human skin before and after exposure to light. Arch Biochem Biophys. 123:468-476.
7. Katiyar, S. K., F. Afaq, K. Azizuddin, and H. Mukhtar. (2001) Inhibition of UVB-induced oxidative stress-mediated phosphorylation of mitogen-activated protein kinase signaling pathways in cultured human epidermal keratinocytes by green tea polyphenol (−)-epigallocatechin-3-gallate. Toxicol Appl Pharmacol. 176:110-117.
8. Hemming, T., K. Jung, and J. Fuchs. (2006) Measurements of UV-generated free radicals/reactive oxygen species (ROS) in skin. Spectrochim Acta A Mol Biomol Spectrosc. 63:840-845.
9. Hancock, J. T., R. Desikan, and S. J. Neill. (2001) Does the redox status of cytochrome C act as a fail-safe mechanism in the regulation of programmed cell death? Free Radic Biol Med. 31:697-703.
10. Pourzand, C., R. D. Watkin, J. E. Brown, and R. M. Tyrrell. (1999) Ultraviolet A radiation induces immediate release of iron in human primary skin fibroblasts: the role of ferritin. Proc Natl Acad Sci USA. 96:6751-6756.
11. Vink, A. A., D. B. Yarosh, and M. L. Kripke. (1996) Chromophore for UV-induced immunosuppression: DNA. Photochem Photobiol. 63:383-386.
12. Larsson, P., E. Andersson, U. Johansson, K. Ollinger, and I. Rosdahl. (2005) Ultraviolet A and B affect human melanocytes and keratinocytes differently. A study of oxidative alterations and apoptosis. Exp Dermatol. 14:117-123.
13. Athar, M., K. P. An, X. Tang, K. D. Morel, A. L. Kim, L. Kopelovich, and D. R. Bickers. (2004) Photoprotective effects of sulindac against ultraviolet B-induced phototoxicity in the skin of SKH-1 hairless mice. Toxicol Appl Pharmacol. 195:370-378.
14. Sen, C. K. (2000) Cellular thiols and redox-regulated signal transduction. Curr Top Cell Regul. 36:1-30.
15. Nishimura, N., V. E. Reeve, H. Nishimura, M. Satoh, and C. Tohyama. (2000) Cutaneous metallothionein induction by ultraviolet B irradiation in interleukin-6 null mice. J Invest Dermatol. 114:343-348.
16. Kramer, M., C. Sachsenmaier, P. Herrlich, and H. J. Rahmsdorf. (1993) UV irradiation-induced interleukin-1 and basic fibroblast growth factor synthesis and release mediate part of the UV response. J Biol. Chem. 268:6734-6741.
17. Balkwill, F., and A. Mantovani. (2001) Inflammation and cancer: back to Virchow? Lancet. 357:539-545.
18. Dannenberg, A. J., and K. Subbaramaiah. (2003) Targeting cyclooxygenase-2 in human neoplasia: rationale and promise. Cancer Cell. 4:431-436.
19. Horwitz, L. D. (2003) Bucillamine: a potent thiol donor with multiple clinical applications. Cardiovasc Drug Rev. 21:77-90.
20. Dhanalakshmi, S., G. U. Mallikarjuna, R. P. Singh, and R. Agarwal. (2004) Silibinin prevents ultraviolet radiation-caused skin damages in SKH-1 hairless mice via a decrease in thymine dimer positive cells and an up-regulation of p53-p21/Cip1 in epidermis. Carcinogenesis. 25:1459-1465.
21. Horwitz, L. D., and N. A. Sherman. (2001) Bucillamine prevents myocardial reperfusion injury. J Cardiovasc Pharmacol. 38:859-867.
22. Amersi, F., S. K. Nelson, X. D. Shen, H. Kato, J. Melinek, J. W. Kupiec-Weglinski, L. D. Horwitz, R. W. Busuttil, and M. A. Horwitz. (2002) Bucillamine, a thiol antioxidant, prevents transplantation-associated reperfusion injury. Proc Natl Acad Sci USA. 99:8915-8920.
23. Stewart, Z. A., and J. A. Pietenpol. (2001) p53 Signaling and cell cycle checkpoints. Chem Res Toxicol. 14:243-263.
24. Maki, C. G., and P. M. Howley. (1997) Ubiquitination of p53 and p21 is differentially affected by ionizing and UV radiation. Mol Cell Biol. 17:355-363.
25. Siegel, D., A. Anwar, S. L. Winski, J. K. Kepa, K. L. Zolman, and D. Ross. (2001) Rapid polyubiquitination and proteasomal degradation of a mutant form of NAD(P)H:quinone oxidoreductase 1. Mol. Pharmacol. 59:263-268.
26. Jaffe, D. R., and G. T. Bowden. (1986) Ionizing radiation as an initiator in the mouse two-stage model of skin tumor formation. Radiat Res. 106:156-165.
27. Jaffe, D., and G. T. Bowden. (1987) Ionizing radiation as an initiator: effects of proliferation and promotion time on tumor incidence in mice. Cancer Res. 47:6692-6696.
28. Liebler, D. C., and J. A. Burr. (2000) Effects of UV light and tumor promoters on endogenous vitamin E status in mouse skin. Carcinogenesis. 21:221-225.
29. Ouhtit, A., H. K. Muller, D. W. Davis, S. E. Ullrich, D. McConkey, and H. N. Ananthaswamy. (2000) Temporal events in skin injury and the early adaptive responses in ultraviolet-irradiated mouse skin. Am J Pathol. 156:201-207.
30. Matsumura, Y., and H. N. Ananthaswamy. (2002) Short-term and long-term cellular and molecular events following UV irradiation of skin: implications for molecular medicine. Expert Rev Mol. Med. 4:1-22.
31. Matsumura, Y., and H. N. Ananthaswamy. (2004) Toxic effects of ultraviolet radiation on the skin. Toxicol Appl Pharmacol. 195:298-308.
32. Halliwell, B., R. Aeschbach, J. Loliger, and O. I. Aruoma. (1995) The characterization of antioxidants. Food Chem. Toxicol. 33:601-617.
33. Kladna, A., H. Y. Aboul-Enein, I. Kruk, T. Michalska, and K. Lichszteld. (2006) Anti-oxidant and pro-oxidant behaviour of bucillamine. Luminescence. 21:90-97.
34. Whitekus, M. J., N. Li, M. Zhang, M. Wang, M. A. Horwitz, S. K. Nelson, L. D. Horwitz, N. Brechun, D. Diaz-Sanchez, and A. E. Nel. (2002) Thiol antioxidants inhibit the adjuvant effects of aerosolized diesel exhaust particles in a murine model for ovalbumin sensitization. J. Immunol. 168:2560-2567.
35. Hiura, T. S., N. Li, R. Kaplan, M. Horwitz, J. C. Seagrave, and A. E. Nel. (2000) The role of a mitochondrial pathway in the induction of apoptosis by chemicals extracted from diesel exhaust particles. J. Immunol. 165:2703-2711.
36. Hikichi, T., F. Mori, M. Nakamura, N. Shishido, M. Sasaki, Y. Horikawa, and A. Yoshida. (2002) Inhibitory effects of bucillamine on increased blood-retinal barrier permeability in streptozotocin-induced diabetic rats. Curr Eye Res. 25:1-7.
37. Mori, F., J. Takahashi, T. Nagaoka, T. Abiko, T. Hikichi, and A. Yoshida. (2006) Inhibitory effect of bucillamine on the increased leukocyte entrapment in the retinal microcirculation of diabetic rats. Jpn J Opthalmol. 50:377-379.
38. Ceconi, C., S. Curello, A. Cargnoni, R. Ferrari, A. Albertini, and O. Visioli. (1988) The role of glutathione status in the protection against ischaemic and reperfusion damage: effects of N-acetyl cysteine. J Mol Cell Cardiol. 20:5-13.
39. De Flora, S., A. Izzotti, F. D'Agostini, and R. M. Balansky. (2001) Mechanisms of N-acetylcysteine in the prevention of DNA damage and cancer, with special reference to smoking-related end-points. Carcinogenesis. 22:999-1013.
40. Iwatani, M., E. Inoue, T. Nakamura, A. Nakajima, M. Hara, T. Tomatsu, N. Kamatani, and H. Yamanaka. (2006) Efficacy profile of bucillamine in rheumatoid arthritis patients in a large observational cohort study, IORRA. Mod Rheumatol. 16:376-380.
41. Munakata, Y., S. Iwata, J. Dobers, T. Ishii, M. Nori, H. Tanaka, and C. Morimoto. (2000) Novel in vitro effects of bucillamine: inhibitory effects on proinflammatory cytokine production and transendothelial migration of T cells. Arthritis Rheum. 43:1616-1623.
42. Schwarz, T., and T. A. Luger. (1989) Effect of UV irradiation on epidermal cell cytokine production. J Photochem Photobiol B. 4:1-13.
43. Dinarello, C. A. (1996) Biologic basis for interleukin-1 in disease. Blood. 87:2095-2147.
44. Maltzman, W., and L. Czyzyk. (1984) UV irradiation stimulates levels of p53 cellular tumor antigen in nontransformed mouse cells. Mol Cell Biol. 4:1689-1694.
45. Fritsche, M., C. Haessler, and G. Brandner. (1993) Induction of nuclear accumulation of the tumor-suppressor protein p53 by DNA-damaging agents. Oncogene. 8:307-318.
46. Shieh, S. Y., M. Ikeda, Y. Taya, and C. Prives. (1997) DNA damage-induced phosphorylation of p53 alleviates inhibition by MDM2. Cell. 91:325-334.
47. Unger, T., T. Juven-Gershon, E. Moallem, M. Berger, R. Vogt Sionov, G. Lozano, M. Oren, and Y. Haupt. (1999) Critical role for Ser20 of human p53 in the negative regulation of p53 by Mdm2. Embo J. 18:1805-1814.
48. Cory, S., and J. M. Adams. (2002) The Bcl2 family: regulators of the cellular life-or-death switch. Nat Rev Cancer. 2:647-656.
49. Isozaki, T., M. Kimura, N. Ikegaya, T. Arai, Y. Fujigaki, A. Hishida, and E. Kaneko. (1992) Bucillamine (a new therapeutic agent for rheumatoid arthritis) induced nephrotic syndrome: a report of two cases and review of the literature. Clin Investig. 70:1036-1042.
50. Yamamoto, T., and H. Yokozeki. (2007) Yellow nails under bucillamine therapy for rheumatoid arthritis: a report of two cases. Rheumatol Int. 27:603-604.

We claim:

1. A method for treatment or prevention of damage to mammalian cells due to exposure to UV-light which comprises the step of administering to an individual susceptible to such damage an effective amount of one or more compounds of formula: 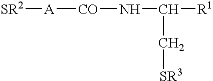
and pharmaceutically acceptable salts and solvates thereof;
where R¹ is selected from a carboxylic acid (COOH or COO—) group, an ester (COOR) or an amide (CON(R′)₂) thereof, where R is alkyl, phenyl, or phenyl-alkyl and each R′, independently, is hydrogen, alkyl, phenyl, or phenyl-alkyl group; R² or R³, independently of one another, are selected from hydrogen, an alkyl group, an alkanoyl group, a phenyl-alkyl group or a phenylcarbonyl group, wherein the alkyl groups, or the phenyl ring of any of these groups is optionally substituted with one or more substituents selected from halogen atoms, alkyl groups, hydroxy groups, alkoxy groups, alkoxyalkyl groups, alkylene groups, alkyleneoxy groups, alkylenedioxy groups, nitro groups, amino groups and alkylamino groups; and A is a lower alkylene group which may be straight chain or branched.

2. The method of claim 1 wherein A is a CH₃CH<, (CH₃)₂C<, —(CH₃)₂C—CH₂—, —(CH₂)₂— or —CH₂— group.

3. The method of claim 1 wherein both of R² and R³ are hydrogens.

4. The method of claim 1 wherein R¹ is a COOH or COO⁻.

5. The method of claim 1 wherein the compound is bucillamine.

6. The method of claim 1 wherein the one or more compounds are administered prior to exposure of the individual to UV-light to prevent damage from UV-light.

7. The method of claim 1 wherein the one or more compounds are administered after exposure of the individual to UV-light to treat damage from UV-light.

8. The method of claim 1 wherein the compound is administered in combination with a pharmaceutically acceptable carrier.

9. The method of claim 1 wherein the compound is administered orally.

10. The method of claim 1 wherein the compound is administered parentally.

11. The method of claim 1 wherein the compound is administered topically to skin that has been or may be exposed to UV light.

12. The method of claim 1 wherein the mammalian cells to be treated or protected are those of the skin.

13. The method of claim 1 wherein the mammalian cells to be treated or protected are those of the eyes.

14. The method of claim 1 wherein the UV light is UVB light.

15. The method of claim 1 which is applied for prevention and treatment of UV-induced acute photodamage.

16. The method of claim 1 which treats or prevents UV-induced edema, erythema or thickening of the skin.

17. The method of claim 1 wherein the one or more compounds of the invention are combined with at least one sunfiltering or sunscreening compound.

18. A sunscreen composition which comprises an effective amount of one or more compounds of formula: 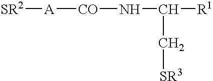
and pharmaceutically acceptable salts and solvates thereof;
where R¹ is selected from a carboxylic acid (COOH or COO—) group, an ester (COOR) or an amide (CON(R′)₂) thereof, where R is alkyl, phenyl, or phenyl-alkyl and each R′, independently, is hydrogen, alkyl, phenyl, or phenyl-alkyl group; R² or R³, independently of one another, are selected from hydrogen, an alkyl group, an alkanoyl group, a phenyl-alkyl group or a phenylcarbonyl group, wherein the alkyl groups, or the phenyl ring of any of these groups is optionally substituted with one or more substituents selected from halogen atoms, alkyl groups, hydroxy groups, alkoxy groups, alkoxyalkyl groups, alkylene groups, alkyleneoxy groups, alkylenedioxy groups, nitro groups, amino groups and alkylamino groups; and A is a lower alkylene group which may be straight chain or branched.

19. The sunscreen composition of claim 18 wherein A is a CH₃CH<, (CH₃)₂C<, —(CH₃)₂C—CH₂—, —(CH₂)₂— or —CH₂— group; both of R² and R³ are hydrogens and R¹ is a COOH or COO⁻.

20. The sunscreen composition of claim 18 wherein the compound of formula I is bucillamine or a salt or solvate thereof.
